(19) United States
(12) Patent Application Publication
(10) Pub. No.: US 2008/0058906 A1
(43) Pub. Date: Mar. 6, 2008

(54) METHODS AND SYSTEMS FOR DELIVERING RADIATION THERAPY TO TREAT DISORDERS IN PATIENTS

(75) Inventors: Charles W. Spangler, Pray, MT (US); Aleksander Rebane, Bozeman, MT (US); Jean-Pierre Laurent, Seattle, WA (US)

Correspondence Address: PERKINS COIE LLP;PATENT-SEA, P.O. BOX 1247, SEATTLE, WA, 98111-1247, US

(73) Assignee: Rasiris, Inc., Bozeman, MT (US)

(21) Appl. No.: 11/675,577

(22) Filed: Feb. 15, 2007

Related U.S. Application Data
(60) Provisional application No. 60/774,332, filed on Feb. 17, 2006.

(51) Int. Cl. A61B 18/20

(52) U.S. Cl. 607/89

(57) ABSTRACT

Methods and systems for delivering radiation therapy to treat disorders in patients are described herein. In one embodiment, a method includes obtaining imaging data of a target in a patient, irradiating the target with a laser beam directed at the target based on the obtained imaging data, and activating a photodynamic therapy agent in the patient with the laser beam. The target can be subcutaneous, cutaneous, or have both subcutaneous and cutaneous portions.

### CROSS-REFERENCE TO RELATED APPLICATION

This application claims the benefit of U.S. Provisional Application No. 60/774,332, filed on Feb. 17, 2006, and entitled METHOD AND SYSTEMS FOR DELIVERING RADIATION THERAPY TO TREAT DISORDERS IN PATIENTS.

U.S. patent application Ser. No. 10/805,683 is incorporated herein by reference.

### TECHNICAL FIELD

The present invention is related to methods and systems for delivering radiation therapy to treat cancer and other disorders in patients.

### BACKGROUND

Cancer is the second leading cause of death among Americans, and the American Cancer Society estimates that more than 1.3 million new cases of cancer were diagnosed in 2005. Approximately 212,930 of these new cases were invasive breast cancer (defined as Stages I-IV) that will cause an estimated 40,870 deaths. Additionally, many more cases of other types of cancer, such as head and neck cancers, occur every year. Early detection of breast cancer and other cancers is critical to successful treatment and enhanced survival rates, as illustrated below in Table 1.

TABLE 1
5-Year Survival Rates for Breast Cancer Patients

| Breast Cancer | 5-Year Relative Survival |
|---|---|
| Stage | Rate |
| 0 | 100% |
| I | 98 |
| II a | 88 |
| II b | 76 |
| III a | 56 |
| III b | 49 |
| IV | 16 |

Many health care professionals recommend that women over the age of 40 undergo annual screening for breast cancer. Unfortunately, the American Cancer Society estimates that only 62% of women over the age of 40 had a mammogram last year. Although recommended, routine mammography has several drawbacks. First, many women experience severe discomfort during a typical mammogram procedure, particularly from breast compression. This discomfort may dissuade some women from undergoing mammogram screening in subsequent years. Second, the results of a mammogram screening may not be accurate. For example, up to 20% of breast cancers are undetected at the yearly mammogram screening stage because the smallest tumor that can be detected is approximately 1-5 mm. Traditional mammography is also plagued by false positives, which can require a follow-up mammogram and/or biopsy. As a result, there exists a need for an improved procedure to accurately detect breast cancer at an early stage without causing discomfort to the patient.

Conventional treatments for breast cancer include surgery, chemotherapy, and/or radiation therapy using a high-intensity beam of ionizing radiation. Because breast cancer varies from person to person, no single treatment may be effective for all patients. Typical surgeries for treating breast cancer include cutting, ablating, or otherwise removing an entire breast or only a portion of a breast where the cancer is located. Surgery, however, is not a viable option for many patients because of the location of cancer. Surgical treatments may also result in complications with anesthesia or infection, and surgical treatments may have long, painful recovery periods. Chemotherapy involves chemically treating the cancer with drugs, which can have many side effects including fatigue, nausea, vomiting, pain in the extremities, hair loss, and anemia. Radiation therapy using ionizing radiation beams is also difficult because the breast may move during the radiation treatment such that healthy tissue is irradiated instead of the lesion. Accordingly, there exists a need for a cancer treatment that avoids or at least minimizes the above-noted drawbacks of conventional treatments.

### BRIEF DESCRIPTION OF THE DRAWINGS

FIG. 1 is a schematic illustration of a system for delivering radiation therapy to treat disorders in patients in accordance with one embodiment of the invention.

FIG. 2 is a flow chart illustrating a method for delivering radiation therapy to treat disorders in patients in accordance with one embodiment of the invention.

FIGS. 3 and 4 illustrate several procedures in one method for delivering radiation therapy to treat breast cancer in patients in accordance with an embodiment of the invention.

FIG. 3 is a schematic illustration of a patient and a portion of the radiation therapy system of FIG. 1.

FIG. 4 is a schematic illustration of a path for irradiating lymph nodes in a patient in accordance with one embodiment of the invention.

FIG. 5 is a schematic illustration of a system for delivering radiation therapy to treat disorders in patients in accordance with another embodiment of the invention.

FIG. 6 is a schematic illustration of a system for delivering radiation therapy to treat disorders in patients in accordance with another embodiment of the invention.

FIG. 7 is a schematic illustration of a system for delivering radiation therapy to treat disorders in patients in accordance with another embodiment of the invention.

FIG. 8 is a schematic illustration of a system for delivering radiation therapy to treat disorders in patients in accordance with another embodiment of the invention.

FIG. 9A a schematic illustration of a system for delivering radiation therapy to treat disorders in patients in accordance with still another embodiment of the invention, and FIG. 9B is a schematic illustration of an imaging unit and treating head that can be used in the system illustrated in FIG. 9A.

FIG. 10 is a schematic illustration of a system for delivering radiation therapy to treat disorders in patients in accordance with yet another embodiment of the invention.

### DETAILED DESCRIPTION

### A. Overview

The following disclosure describes several embodiments of methods and systems for delivering radiation therapy to treat disorders in patients. An embodiment of one such method includes obtaining imaging data of a target in a patient, irradiating the target with a radiation directed at the target based on the obtained imaging data, and activating a photodynamic therapy agent in the patient with the radiation. The radiation is transcutaneously transmitted from the radiation source or radiation delivery device to subcutaneous targets, or the radiation is transmitted from an external site for cutaneous targets. The target, therefore, can be subcutaneous, cutaneous, or have both subcutaneous and cutaneous portions. Irradiating the target may include scanning the laser beam across the target or irradiating the entire target simultaneously. The radiation source can be a laser, a light emitting diode (LED), or a lamp (e.g., incandescent and/or fluorescent).

In another embodiment, a method includes introducing into a patient an agent including a targeting component for directing the agent to a target in the patient, imaging the target by irradiating the target with radiation having a first wavelength to activate an imaging component of the agent, and activating a photodynamic therapy component of the agent by applying radiation having a second wavelength to the target. In several applications, the activated imaging component of the agent fluoresces at the target. Imaging the target can include capturing the fluorescence of the imaging component at the target and generating a three-dimensional image of the target based on the captured fluorescence.

In another embodiment, a method includes irradiating a target in a patient with radiation having a first wavelength, generating a digital three-dimensional image of the target, and irradiating the target based on the three-dimensional image with radiation having a second wavelength different than the first wavelength. Irradiating the target with radiation having the second wavelength can include irradiating the target with a pulsed laser capable of initiating efficient two-photon absorption or other suitable lasers.

Another aspect of the invention is directed to systems for delivering radiation therapy to treat disorders in patients. In one embodiment, a system includes an imaging unit for capturing an image of a target in a patient, a laser for irradiating the target and activating a photodynamic therapy agent in the patient, and a controller operably coupled to the imaging unit and the laser for operating the laser to irradiate the target based on the captured image of the target. The laser is configured to generate a laser beam along a beam path, and the system can further include an optical element assembly aligned with the beam path for redirecting and/or conditioning the laser beam. The system may further include a second laser for irradiating the target and activating an imaging agent at the target.

In another embodiment, a system includes a first radiation delivery device for irradiating a target in a patient with radiation having a first wavelength, an imaging unit for capturing a digital image of the target, a second radiation delivery device for irradiating the target with radiation having a second wavelength different than the first wavelength, and a controller operably coupled to the second radiation delivery device and the imaging unit for irradiating the target with radiation having the second wavelength based on the captured digital image.

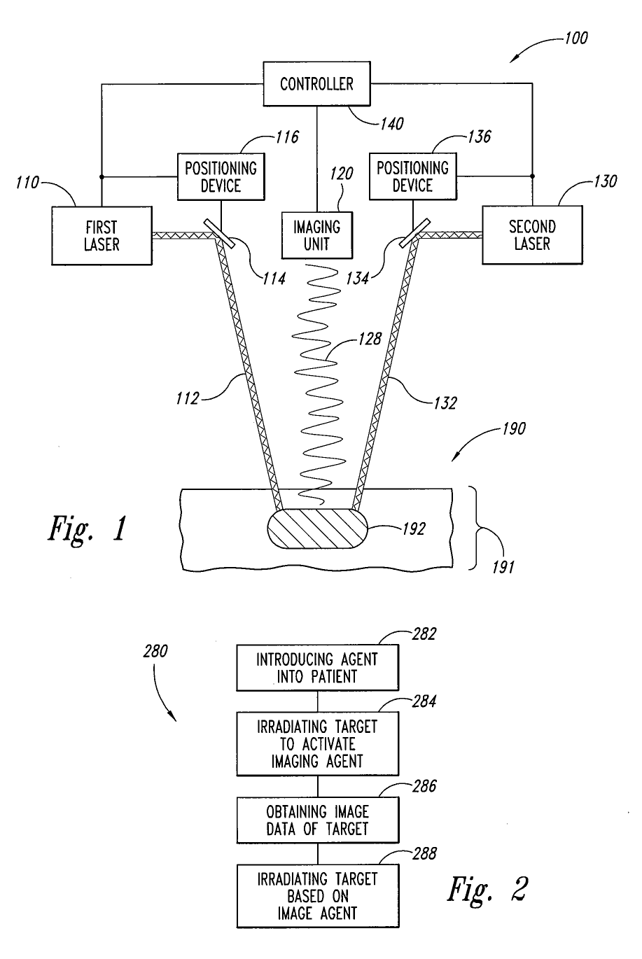

Specific details of several embodiments of the invention are described below with reference to systems and methods for delivering radiation therapy to treat cancer, but in other embodiments the systems and methods can be used to treat other diseases and disorders. Several details describing well-known structures or processes often associated with delivering radiation therapy are not set forth in the following description for purposes of brevity and clarity. Also, several other embodiments of the invention can have different configurations, components, or procedures than those described in this section. A person of ordinary skill in the art, therefore, will accordingly understand that the invention may have other embodiments with additional elements, or the invention may have other embodiments without several of the elements shown and described below with reference to FIGS. 1-10. Where the context permits, singular or plural terms may also include the plural or singular term, respectively. Moreover, unless the word “or” is expressly limited to mean only a single item exclusive from other items in reference to a list of at least two items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same features and/or types of other features and components are not precluded.

### B. Embodiments of Systems for Delivering Radiation Therapy

FIG. 1 is a schematic illustration of a system 100 for delivering radiation therapy to treat disorders in patients in accordance with one embodiment of the invention. The system 100 irradiates an area of a patient 190 containing a target 192 to activate an imaging agent at the target 192. The activated imaging agent at the target 192 fluoresces or emits another type of radiation. The system 100 captures the radiation and generates an image of the target 192 from the radiation. Based on the imaging data, the system 100 selectively irradiates the target 192 to activate a photodynamic agent, which treats a specific disorder (e.g., cancer) in the patient 190. In the case of treating cancer, for example, the target can be in the breast, head, neck or other affected area of the patient.

The illustrated system 100 includes a first laser 110 (shown schematically) for producing a first laser beam 112, a first optical element assembly 114 for directing and/or conditioning the first laser beam 112, and a positioning device 116 (shown schematically) for moving and/or positioning the optical element assembly 114 to direct the first laser beam 112 toward a desired area of the patient 190. The first laser 110 can be a diode laser, a gas laser, a solid state laser, a dye laser, a fiber laser, a pulsed laser capable of initiating efficient two-photon absorption, or another suitable laser for generating the first laser beam 112. In either case, the first laser 110 is configured to generate the first laser beam 112 with a wavelength selected to penetrate tissue and activate an imaging agent in the patient 190. For example, in applications in which the imaging agent is Indotricarbocyanine, the first laser beam 112 can have a wavelength of approximately 748 nanometers. In other embodiments, the first laser 110 can generate a variable-frequency laser beam, and/or the first laser beam 112 can have a different wavelength in the near-infrared range or in other suitable ranges to penetrate the tissue and activate the imaging agent in the patient 190. In additional embodiments, the system 100 may not include the first laser 110, but rather a different type of radiation source to activate the imaging agent in the patient 190. For example, the radiation source for activating the imaging agent or other component of the drug may be an LED array or a lamp in lieu of or in addition to a laser.

The first optical element assembly 114 conditions the first laser beam 112 and directs the beam 112 from the first laser 110 to a desired area of the patient 190. The first optical element assembly 114 can include a collimator, lenses, mirrors, beam splitters, and/or other suitable optical members that redirect and/or condition the first laser beam 112. The positioning device 116 is operably coupled to the first optical element assembly 114 for moving one or more components of the assembly 114 to direct the first laser beam 112 toward a desired area of the patient 190. For example, in several applications, the positioning device 116 can move the first optical element assembly 114 to scan the first laser beam 112 across an area of the patient 190 that includes the target 192. In other embodiments, the system 100 may include another positioning device for moving the first laser 110 or the patient 190 in lieu of or in addition to the positioning device 116.

The illustrated system 100 further includes an imaging unit 120 for capturing radiation 128 emitted by the imaging agent in the patient 190 and a controller 140 (shown schematically) operably coupled to the first laser 110, the positioning device 116, and the imaging unit 120. The imaging unit 120 captures and digitizes the radiation 128 emitted by the activated imaging agent at the target 192 to generate a three-dimensional image of the target 192. The imaging unit 120 can include a CCD camera, a CT scanner, an MRI machine, an X-ray apparatus, or another suitable imaging device. Suitable CCD cameras include the Pixis camera manufactured by Roper Scientific of Trenton, N.J.; the Maestro manufactured by CRI Inc. of Woburn, Mass.; the Image Station 4000 mM manufactured by Kodak Inc. of Rochester, N.Y.; and the D-Light fluorescence system manufactured by Carl Storz of Tuttlingen, Germany. The imaging unit 120, for example, may have time-gated cameras that detect the emission and scattering of the radiation caused by the imaging component or other component of the drug. Such time-gated detection can occur during, between or after illumination pulses. The imaging unit 120 may further include lenses and/or filters to separate the radiation 128 emitted by the imaging agent from the excitation light. For example, in one embodiment, the imaging unit 120 includes two CCD cameras having different filters such that one camera captures the excitation light and the other camera captures the radiation 128 emitted from the imaging agent. In either case, the imaging unit 120 and/or the controller 140 generates a three-dimensional image of the target 192 based on the radiation 128 emitted from the imaging agent at the target 192. The system 100 may further include a high-resolution display (not shown) so that an oncologist and/or technician can observe the image.

The system 100 illustrated in FIG. 1 further includes a second laser 130 (shown schematically) for producing a second laser beam 132, a second optical element assembly 134 for directing and/or conditioning the second laser beam 132, and a positioning device 136 for moving and/or positioning the second optical element assembly 134 to direct the second laser beam 132 toward a desired area of the patient 190. The second laser 130 is configured to produce the second laser beam 132 with a wavelength selected to activate a photodynamic therapy agent at the target 192. In several applications, the second laser 130 can be a pulsed laser capable of initiating efficient two-photon absorption that produces a laser beam in the near-infrared range (e.g., 750-850 nanometers). For example, in one embodiment, the second laser 130 has the following parameters:

Laser wavelength: λ=750-900 nm

Laser temporal pulse length: τₚ=100 fs-1 ps

Laser pulse energy: P=1.0-10 mJ

Laser surface spot area: S=1 cm²

Laser pulse repetition rate: R=1-10 kHz

In other embodiments, however, the second laser 130 may have different parameters. Another suitable embodiment of the second laser 130 can provide beams with wavelengths of approximately 800-1,500 nanometers and pulse durations of 1 ps to 100 ns. Suitable lasers may include the Libra and Legend lasers manufactured by Coherent Inc. of Santa Clara, Calif.; the CPA-series lasers manufactured by Clark-MXR Inc. of Dexter, Mich.; the Integra lasers manufactured by Quantronix of East Setauket, N.Y.; the Eclipse laser manufactured by Spectra-Physics Lasers of Mountain View, Calif.; the μjewel laser manufactured by IMRA America of Ann Arbor, Mich.; the Fortis laser manufactured by Time-Bandwidth of Zurich, Switzerland; and the femtoRegen laser manufactured by High-Q Laser of Hohenems, Austria. In other embodiments, the second laser 130 may not be a pulsed laser capable of initiating efficient two-photon absorption, and/or the system 100 may include a single laser capable of generating the first and second laser beams 112 and 132 in lieu of the first and second lasers 110 and 130.

The second optical element assembly 134 conditions the second laser beam 132 and directs the beam 132 from the second laser 130 toward the target 192 of the patient 190. The second optical element assembly 134 also focuses the second laser beam 132 to a desired depth within the patient 190. The second optical element assembly 134 can include a collimator, lenses, mirrors, and/or other optical members that redirect, condition, and/or focus the second laser beam 132. The positioning device 136 is coupled to the second optical element assembly 134 for moving one or more components of the assembly 134, and the controller 140 is operably coupled to the second laser 130 and the positioning device 136 to direct and focus the second laser beam 132 toward the target 192 in the patient 190 based on the imaging data. As a result, the second laser beam 132 can activate the photodynamic therapy agent in the target 192. The optical assembly further include an optical fiber, fiber bundle or light pipe to transmit the laser beam to toward the target. For example, a high peak intensity pulsed laser beam (e.g., >10⁹ W) may be transmitted through a photonic fiber with a high damage threshold.

### C. Embodiments of Methods for Treating Disorders in Patients with Radiation Therapy

FIG. 2 is a flow chart illustrating a method 280 for delivering radiation therapy to treat disorders in patients in accordance with one embodiment of the invention. The method 280 is particularly well suited for treating breast cancer, skin cancer, head cancers, neck cancers and other types of cutaneous and subcutaneous disorders in patients. The illustrated method includes an introduction process 282, a first irradiation process 284, an imaging process 286, and a second irradiation process 288. Referring to both FIGS. 1 and 2, the introduction process 282 includes introducing an agent into the patient 190. The agent can be injected (e.g., via an intravenous injection), swallowed, or introduced into the patient 190 through some other suitable method. In several applications, the agent can be a trifunctional compound having a targeting component, an imaging component, and a photodynamic therapy component. The targeting component directs the agent to the target 192 in the patient 190 based on the particular location, cell type, or other characteristic of the target 192. For example, in applications in which the patient 190 has cancerous cells, the targeting component can direct the trifunctional agent to these cells by having an affinity to rapidly dividing cells or other properties of cancerous cells. The imaging component can be Indotricarbocyanine or other suitable compounds that emit fluorescence or another type of radiation when activated. The imaging component may be activated by a specific wavelength of non-ionizing radiation or some other method. The photodynamic therapy component can initiate the death of certain cells (e.g., cancer cells) after activation by a specific wavelength of radiation or another method. Suitable agents and components are described in U.S. patent application Ser. No. 10/805,683, which is attached hereto as Appendix A. In other embodiments, the three components can be introduced into the patient 190 separately. In additional embodiments, the agent can be a bifunctional agent having only imaging and photodynamic therapy components. In either case, after introducing the agent into the patient 190, the method 280 typically includes a waiting period of up to a few days (e.g., 96 hours) for allowing the agent to accumulate at the target 192.

The first irradiation process 284 includes generating the first laser beam 112 with the first laser 110 and directing the first laser beam 112 at a section of tissue 191 containing the target 192 to activate the imaging component. Although the precise location, shape, and size of the target 192 in the patient 190 is often unknown, the general location of the section of tissue 191 containing the target 192 is known. As a result, the second laser beam 132 irradiates the section of tissue 191 known to contain the target 192. In one embodiment, the positioning device 116 can move the first optical element assembly 114 to raster scan the first laser beam 112 across the section of tissue 191 to irradiate and activate the imaging component of the agent at the target 192. One benefit of scanning the first laser beam 112 across the section of tissue 191 is that the imaging unit 120 can generate a three-dimensional image of the target 192 based on finite-element diffuse light recovery known in the art. Alternatively, the first laser beam 112 can have a spot size configured to simultaneously irradiate at least approximately the entire section of the tissue 191 containing the target 192. In either case, the first laser beam 112 activates the imaging component of the agent at the target 192.

The imaging process 286 includes capturing and digitizing the radiation 128 emitted from the activated imaging component of the agent to generate a three-dimensional image of the target 192. This may require filtering the radiation received from the patient 190 to separate the radiation 128 emitted by the imaging component from the excitation radiation. For example, in applications in which the imaging component is Indotricarbocyanine and emits fluorescence at approximately 780 nanometers, the imaging unit 120 filters out other wavelengths of radiation.

The second irradiation process 288 includes irradiating the target 192 to activate the photodynamic therapy component based on the data obtained from the imaging process 286. Specifically, the imaging data provides information regarding the location, size, shape, and/or other characteristics of the target 192. The controller 140 receives this imaging data from the imaging unit 120 and operates the second laser 130 and the positioning device 136 to aim and focus the second laser beam 132 so that the beam 132 irradiates the target 192 and activates the photodynamic therapy component. For example, the positioning device 136 can raster scan the second laser beam 132 across the target 192. Alternatively, the controller 140 can operate the second laser 130 and the positioning device 136 to irradiate at least approximately the entire target 192 simultaneously. In other embodiments, the controller 140 may not automatically operate the positioning device 136, but rather a technician or oncologist can manually aim and/or focus the second laser beam 132 based on the imaging data. In several applications, the second irradiation process 288 occurs after the first irradiation process 284. However, in other applications, the first and second laser beams 112 and 132 may irradiate the patient 190 concurrently. For example, the first laser beam 112 may scan across the section of tissue 191 and each time the beam 112 irradiates a portion of the target 192 and the imaging unit 120 detects the target 192, the controller 140 can operate the second laser 130 to irradiate that portion of the target 192. In either case, the activated photodynamic therapy component can initiate the death of particular cells at the target 192 or otherwise treat a disorder at the target 192. For example, cancer cells may be destroyed by apoptosis, necrosis and/or autophagy. In additional embodiments in which the method 280 is used to detect and not necessarily treat cancer or another disorder, the method 280 may not include the second irradiation process 288.

The efficacy of the treatment can be determined by one or more follow-up sessions in which a portion of the method 280 is repeated. For example, in one follow-up session the introduction, first irradiation, and imaging processes 282, 284, and 286 can be performed. If the tumor or other disorder at the target 192 has been destroyed, the imaging unit 120 should not detect a concentration of the agent at the target 192.

One feature of the method 280 described above with reference to FIGS. 1 and 2 is that the method 280 includes introducing into a patient an agent with (a) a targeting component that directs the agent to particular cells (e.g., cancerous cells), and (b) an imaging component that emits radiation from the cells. As a result, when the specific cells are present in the patient, the area of the patient with the cells will have a higher concentration of the agent than the rest of the patient. An advantage of this feature is that the method 280 can be used to detect cancer and/or other disorders at early stages. Early detection of cancer and some other disorders is critical to successful treatment and enhanced survival rates. Another advantage of this feature is that the method 280 is not expected to cause significant discomfort to the patient.

Another feature of the method 280 described above with reference to FIGS. 1 and 2 is that the method 280 includes activating a photodynamic therapy component of the agent with the second laser beam 132. The activated photodynamic therapy component initiates the death of certain cells or otherwise treats a disorder in the patient. An advantage of this feature is that the method 280 treats cancer and/or other disorders with noninvasive procedures. As a result, the risks of infection and other complications associated with invasive procedures are minimized or eliminated. Moreover, a patient can receive treatment for cancer or another disorder as an outpatient procedure, which reduces the costs associated with a hospital stay and the inconvenience to the patient.

Still another feature of the system 100 is that it is particularly well suited for treating different types of cancer with one apparatus. The imaging system 120 and the therapy radiation delivery system can be operated using different imaging illumination and PDT radiation modalities that can be optimized for achieving particular cancer cell death pathways. This increases the flexibility of the system such that clinics do not need to have separate equipment that is limited to treating only certain indications.

One feature of the system 100 described above with reference to FIGS. 1 and 2 is that the controller 140 includes a computer-readable medium having instructions for operating the second laser 130 and the second optical element assembly 134 to irradiate the target 192 based on the imaging data. As a result, the system 100 can accurately and precisely aim and focus the second laser beam 132 at the target 192. An advantage of this feature is that the system 100 can irradiate the target 192 without unnecessarily exposing the surrounding tissue to radiation.

### D. Embodiments of Methods for Treating Breast Cancer in Patients with Radiation Therapy

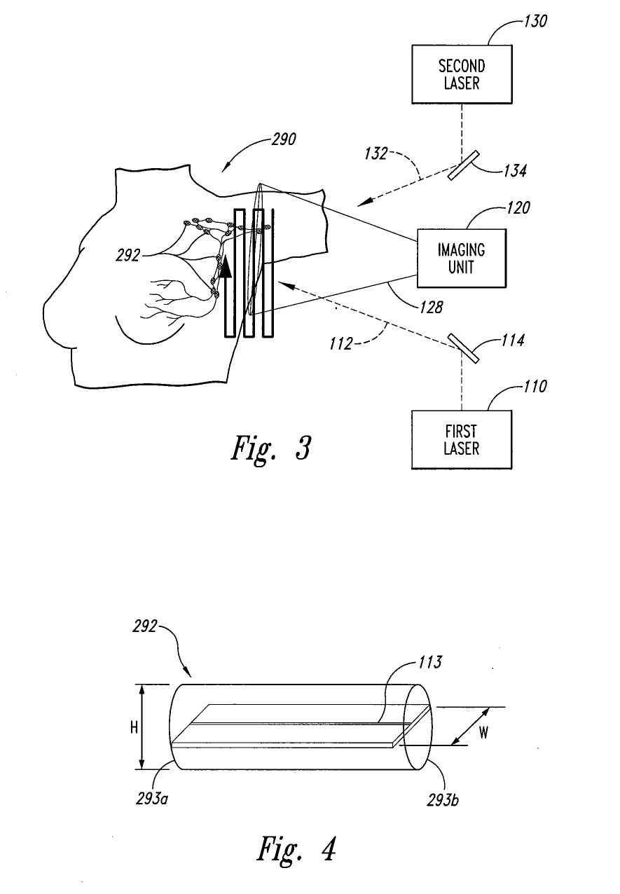

FIGS. 3 and 4 illustrate several procedures in one method for delivering radiation therapy to treat breast cancer in patients in accordance with one embodiment of the invention. For example, FIG. 3 is a schematic illustration of a patient 290 and a portion of the radiation therapy system 100. The method includes introducing a trifunctional agent into the patient 290. The trifunctional agent includes a targeting component specifically configured to carry the agent to the cancerous cells in the lymph nodes 292 proximate to the breast of the patient 290. After the agent has reached the lymph nodes 292, the first laser beam 112 irradiates the area of the patient 290 containing the lymph nodes 292 to activate the imaging component of the agent. The system 100 can raster scan the first laser beam 112 across the area of the patient 290 containing the lymph nodes 292 as illustrated in FIG. 3, or irradiate the entire area simultaneously. In either case, the activated imaging component of the agent at each of the lymph nodes 292 fluoresces, and the fluorescence 128 from the imaging component at each lymph node 292 is captured and digitized by the imaging unit 120. The precise size, shape, and location of the lymph nodes 292 can be determined from the imaging data. Next, the second laser 130 irradiates each lymph node 292 to activate the photodynamic therapy component of the agent at the lymph nodes 292 based on the imaging data.

FIG. 4 is a schematic illustration of a path for irradiating each lymph node 292 in accordance with one embodiment of the invention. The inventors have observed that the effects of a single point of treatment extend approximately 0.5 cm in all directions. Because a typical lymph node 292 has a height H and width W of approximately 1 cm, the system 100 can irradiate an entire lymph node 292 by focusing the second laser beam 132 at a depth corresponding to the middle of the lymph node 292 and moving the beam 132 along a single linear path 113 between the first and second ends 293a-b of the lymph node 292. In other embodiments, the system 100 can irradiate the lymph nodes 292 with a different method.

### E. Additional Embodiments of Systems for Delivering Radiation Therapy

FIG. 5 is a schematic illustration of a system 200 for delivering radiation therapy to treat disorders in patients in accordance with another embodiment of the invention. The system 200 is generally similar to the system 100 described above with reference to FIG. 1. For example, the illustrated system 200 includes a first laser 110 (shown schematically) for generating a first laser beam 112, an imaging unit 120 (shown schematically) for capturing radiation emitted by the imaging agent in the patient 190, a second laser 130 (shown schematically) for generating a second laser beam (not shown), and a controller 140 (shown schematically) operably coupled to the first laser 110, the imaging unit 120, and the second laser 130. The illustrated system 200, however, includes a single optical element assembly 214 for conditioning, directing, and/or focusing the first and second laser beams 112. The optical element assembly 214 may include a collimator, lenses, mirrors, beam splitters, and/or other suitable optical members that redirect, condition, and/or focus the laser beams. The illustrated optical element assembly 214 is movable in at least a direction X so that the assembly 214 is properly positioned to direct (a) the first laser beam 112 from the first laser 110 to a desired area of the patient 190, and (b) the second laser beam from the second laser 130 to the target 192 in the patient 190. In additional embodiments, the optical element assembly 214 may not be movable.

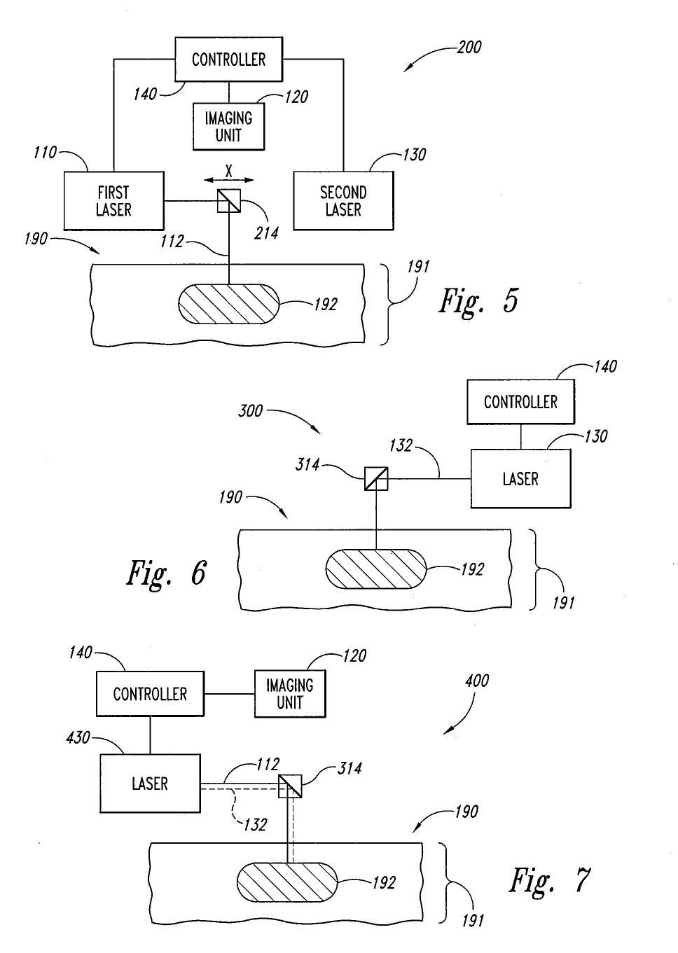

FIG. 6 is a schematic illustration of a system 300 for delivering radiation therapy to treat disorders in patients in accordance with another embodiment of the invention. The system 300 is generally similar to the system 100 described above with reference to FIG. 1. For example, the illustrated system 300 includes a laser 130 (shown schematically) for generating a laser beam 132, an optical element assembly 314 for conditioning, directing, and/or focusing the laser beam 132, and a controller 140 (shown schematically) operably coupled to the laser 130. The illustrated system 300, however, does not include an integral imaging unit. Rather, the controller 140 (a) receives imaging data from an external imaging unit (e.g., a MRI scan, a CT scan, a PET scan or an X-ray), and (b) operates the laser 130 to irradiate the target 192 in the patient 190 based on the imaging data received from the external imaging unit and external fiducials or the guidance of internal markers. The optical element assembly 314 includes a collimator, lenses, mirrors, beam splitters, and/or other suitable optical members that redirect, condition, and/or focus the laser beam 132. The laser 130 can be a pulsed laser capable of initiating efficient two-photon absorption or another suitable device.

FIG. 7 is a schematic illustration of a system 400 for delivering radiation therapy to treat disorders in patients in accordance with another embodiment of the invention. The system 400 is generally similar to the system 100 described above with reference to FIG. 1. For example, the illustrated system 400 includes a laser 430 (shown schematically) for generating a laser beam, an optical element assembly 314 for conditioning, directing, and/or focusing the laser beam, an imaging unit 120 (shown schematically) for capturing radiation emitted by the imaging agent in the patient 190, and a controller 140 (shown schematically) operably coupled to the laser 430 and the imaging unit 120. The illustrated laser 430, however, can generate laser beams with different wavelengths. For example, the laser 430 can generate a first laser beam 112 having a first wavelength for activating the imaging agent and a second laser beam 132 having a second wavelength different than the first wavelength for activating the photodynamic therapy agent. In other embodiments, the system 400 may include two lasers rather than one laser.

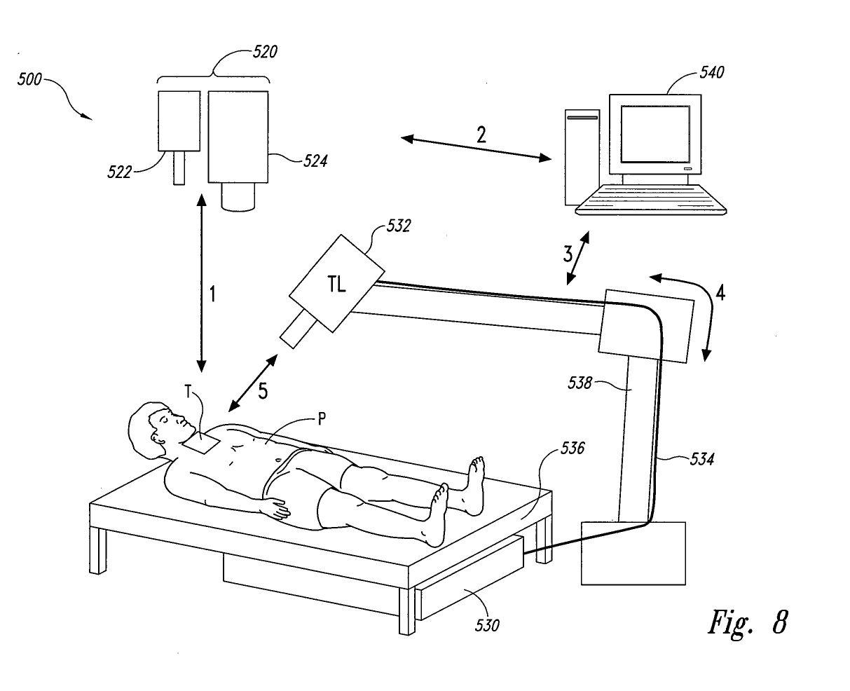

FIG. 8 is schematic illustration of a system 500 for delivering radiation therapy to treat disorders in patients in accordance with another embodiment of the invention. The system 500 is a further embodiment of the system 100 described above with reference to FIG. 1. In this embodiment, the system 500 includes an imaging unit 520 having an imaging radiation source 522 and a detector 524. The imaging radiation source 522 can be a first laser that directs imaging radiation toward a target T of a patient P. The detector 524 can be a camera or other suitable radiation detector. The system 500 further includes a treatment radiation source 530, which can be a second having a suitable wavelength or other property for activating the therapy component of the drug. The system 500 further includes a treating head 532 and a transmission line 534 between the treatment radiation source 530 and the treating head 532. The treating head 532, for example, can be an optical assembly for conditioning, directing, and/or focusing a laser beam generated by the treatment radiation source 530. The transmission line 534 can be an optical fiber, a fiber bundle or a light pipe. In the illustrated embodiment of FIG. 8, the treatment radiation source 530 is attached to a table 536 upon which the patient P is positioned, and the treating head 532 is mounted to a robot 538. The robot 538 can be a robotic arm that has up to 6° of freedom for positioning the treating head 532 at a desired location relative to the target T and the patient P. In one embodiment, the imaging unit 520 is located apart from the treating head 532. In other embodiments, the imaging unit 520 and the treating head 532 can both be mounted to the robot 538. The system 500 further includes a controller operatively coupled to the imaging unit 520, the treatment radiation source 530, the treating head 532 and the robot 538. The controller 540 can be coupled to the other components of the system 500 using electrical lines, optical lines, and/or wireless transmission modalities.

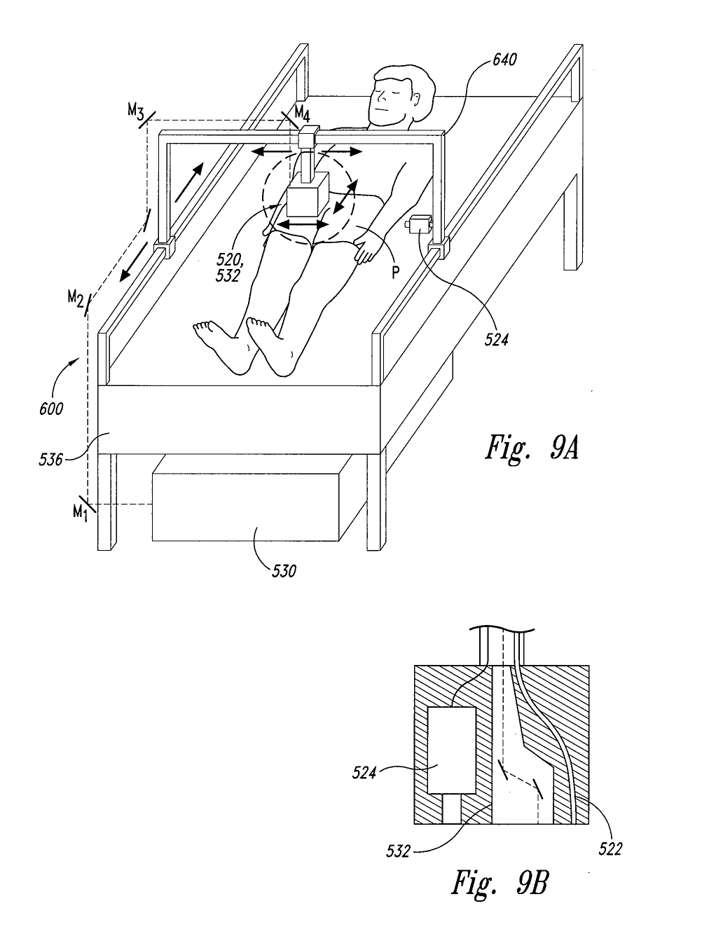

FIG. 9A a schematic illustration of a system 600 for delivering radiation therapy to treat disorders in patients in accordance with yet another embodiment of the invention. The system 600 is generally similar to the system 100 and the system 500 described above, and thus like reference numbers refer to like components in FIGS. 1-9A. The system 600 accordingly includes the imaging unit 520, the treatment radiation source 530, the treating head 532, and the table 536. In this embodiment, the system 600 includes a gantry 640 to which the image unit 520 and the treating head 532 are mounted. The gantry is accordingly operated to position the image unit 520 and the treating head 532 relative to the patient P.

FIG. 9B schematically illustrates an embodiment of the image unit 520 and the treating head 532 that are packaged together. As such, the imaging radiation source 522, at least one detector 524 and the treating head 532 are held in position relative to each other and mounted to the gantry 640 (FIG. 9A).

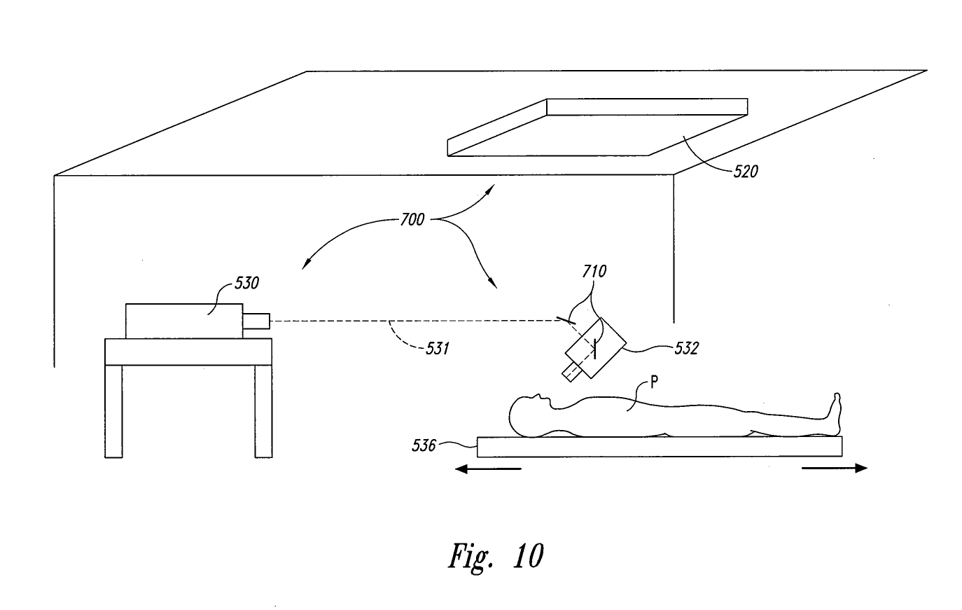

FIG. 10 is a schematic illustration of a system 700 for delivering radiation therapy to treat disorders in patients in accordance with still another embodiment of the invention. The system 700 is generally similar to the system 500 described above with reference to FIG. 8, and thus like reference numbers refer to like components in FIGS. 8 and 10. In this embodiment, the treatment radiation source 530 is spaced apart from the table 536. The system 700 can further include a light guide, such as a mirror, that directs the laser beam 531 to the treating head 532. The laser beam 531 can propagate through the air from the treatment radiation source 530 to the mirror 710. In other embodiments, the mirror 710 can be eliminated and the laser beam 531 can be transmitted through a fiber optic, an optical bundle, or a light pipe.

From the foregoing, it will be appreciated that specific embodiments of the invention have been described herein for purposes of illustration, but that various modifications may be made without deviating from the spirit and scope of the invention. For example, although the targets illustrated in FIGS. 1-10 are subcutaneous targets, in other embodiments, the targets can be cutaneous targets or targets with both cutaneous and subcutaneous portions. Additionally, the imaging modality may also include detecting specific absorption and/or scattering of the radiation caused by the imaging component and/or the PDT sensitizer component of the drug administered to the patient. Accordingly, the invention is not limited except as by the appended claims.

I/We claim:

1. A method for delivering radiation therapy to treat a disorder in a patient, the method comprising:
obtaining imaging data of a target in the patient;
irradiating the target with a laser beam directed at the target based on the obtained imaging data; and
activating a photodynamic therapy agent in the patient with the laser beam.

2. The method of claim 1 wherein:
the photodynamic therapy agent is a component of a multifunctional agent;
the method further comprises introducing the multifunctional agent into the patient, the multifunctional agent further comprising a targeting component for directing the multifunctional agent to the target in the patient;
irradiating the target with the laser beam comprises (a) applying radiation having a first wavelength at the target with a pulsed laser capable of initiating efficient two-photon absorption, and (b) scanning the laser beam across at least a portion of the target;
the method further comprises irradiating the target with radiation having a second wavelength different than the first wavelength to activate an imaging component of the multifunctional agent so that at least a portion of the imaging component at the target fluoresces; and
obtaining imaging data comprises capturing a digital three-dimensional image of the imaging component fluorescing.

3. The method of claim 1 wherein obtaining imaging data comprises capturing a digital three-dimensional image of the target.

4. The method of claim 1 wherein irradiating the target comprises applying radiation with a pulsed laser capable of initiating efficient two-photon absorption.

5. The method of claim 1 wherein irradiating the target comprises focusing the laser beam at the target based on the obtained imaging data.

6. The method of claim 1 wherein irradiating the target comprises automatically scanning the laser beam across at least a portion of the target.

7. The method of claim 1 wherein:
the photodynamic therapy agent is a component of a multifunctional agent; and
the method further comprises introducing the multifunctional agent into the patient, the multifunctional agent further comprising a targeting component for directing the multifunctional agent to the target in the patient.

8. The method of claim 1 wherein:
irradiating the target with the laser beam comprises applying radiation having a first wavelength to the target; and
the method further comprises irradiating the target with radiation having a second wavelength different than the first wavelength to activate an imaging agent.

9. The method of claim 1 wherein:
irradiating the target with the laser beam comprises applying radiation having a first wavelength to the target; and
the method further comprises irradiating the target with radiation having a second wavelength different than the first wavelength to activate an imaging agent while obtaining imaging data of the target.

10. The method of claim 1 wherein:
irradiating the target with the laser beam comprises applying radiation having a first wavelength to the target;
the method further comprises irradiating the target with radiation having a second wavelength different than the first wavelength to activate an imaging agent so that at least a portion of the imaging component at the target fluoresces; and
obtaining imaging data comprises capturing an image of the imaging component at the target fluorescing.

11. The method of claim 1 wherein:
irradiating the target comprises applying radiation to the target with a first laser beam; and
the method further comprises irradiating the target with a second laser beam while irradiating the target with the first laser beam.

12. The method of claim 1 wherein obtaining imaging data comprises capturing a digital image with a camera.

13. The method of claim 1 wherein activating the photodynamic therapy agent comprises activating a photodynamic therapy component of a multifunctional agent.

14. The method of claim 1 wherein obtaining imaging data of the target comprises capturing imaging data of a subcutaneous target.

15. A method for delivering radiation therapy to treat a disorder in a patient, the method comprising:
capturing a digital three-dimensional image of a target in the patient; and
automatically scanning a pulsed laser beam capable of initiating efficient two-photon absorption across the target with a computer based on the captured three-dimensional image.

16. The method of claim 15 wherein automatically scanning the pulsed laser beam comprises irradiating the target with the pulsed laser beam capable of initiating efficient two-photon absorption.

17. The method of claim 15 wherein automatically scanning the pulsed laser beam comprises focusing the laser beam at the target based on the captured digital three-dimensional image.

18. The method of claim 15 wherein capturing the digital three-dimensional image comprises obtaining imaging data of a subcutaneous target in the patient.

19. The method of claim 15 wherein automatically scanning the pulsed laser beam comprises activating a photodynamic therapy agent in the patient with the laser beam.

20. The method of claim 15, further comprising introducing into the patient an agent including a targeting component for directing the agent to the target in the patient.

21. The method of claim 15 wherein:
automatically scanning the pulsed laser beam comprises irradiating the target with radiation having a first wavelength; and
the method further comprises irradiating the target with radiation having a second wavelength different than the first wavelength to activate an imaging agent in the patient.

22. The method of claim 15 wherein:
automatically scanning the pulsed laser beam comprises irradiating the target with radiation having a first wavelength;
the method further comprises irradiating the target with radiation having a second wavelength different than the first wavelength to activate an imaging agent in the patient so that at least a portion of the imaging agent at the target fluoresces; and
capturing the digital three-dimensional image comprises obtaining imaging data of the imaging agent fluorescing.

23. A method for delivering radiation therapy to treat a disorder in a patient, the method comprising:
introducing into the patient an agent including a targeting component for directing the agent to a non-ionizing target in the patient;
imaging the target by irradiating the target with non-ionizing radiation having a first wavelength to activate an imaging component of the agent; and
applying radiation having a second wavelength to the target to activate a photodynamic therapy component of the agent.

24. The method of claim 23 wherein imaging the target further comprises capturing an image of a subcutaneous target.

25. The method of claim 23 wherein imaging the target further comprises generating a digital three-dimensional image of the target.

26. The method of claim 23 wherein:
imaging the target further comprises applying non-ionizing radiation to the target with a first laser beam; and
applying radiation having the second wavelength comprises irradiating the target with a second laser beam.

27. The method of claim 23 wherein applying radiation having the second wavelength comprises irradiating the target with a pulsed laser capable of initiating efficient two-photon absorption.

28. The method of claim 23 wherein applying radiation having the second wavelength comprises irradiating the target with a laser beam directed at the target based on the imaged target.

29. The method of claim 23 wherein imaging the target further comprises:
scanning a laser beam having the first wavelength across the target; and
generating the laser beam with a pulsed laser capable of initiating efficient two-photon absorption.

30. The method of claim 23 wherein imaging the target further comprises:
activating the imaging component of the agent so that at least a portion of the imaging agent at the target fluoresces; and
capturing an image of the imaging agent fluorescing.

31. The method of claim 23 wherein applying radiation having the second wavelength comprises:
automatically scanning a laser beam having the second wavelength across the target; and
focusing the laser beam at the target based on the imaged target.

32. A method for delivering radiation therapy to treat a disorder in a patient, the method comprising:
irradiating a target in the patient with radiation having a first wavelength;
generating a digital three-dimensional image of the target; and
irradiating the target based on the three-dimensional image with radiation having a second wavelength different than the first wavelength.

33. The method of claim 32 wherein:
irradiating the target with radiation having the first wavelength comprises applying radiation to the target with a first laser beam; and
irradiating the target with radiation having the second wavelength comprises applying radiation to the target with a second laser beam.

34. The method of claim 32 wherein irradiating the target with radiation having the second wavelength comprises applying radiation to the target with a pulsed laser capable of initiating efficient two-photon absorption.

35. The method of claim 32 wherein irradiating the target with radiation having the second wavelength comprises activating a photodynamic agent in the patient.

36. The method of claim 32, further comprising introducing into the patient an agent including a targeting component for directing the agent to the target in the patient.

37. The method of claim 32 wherein irradiating the target with radiation having the first wavelength comprises activating an imaging agent in the patient.

38. The method of claim 32 wherein irradiating the target in the patient with radiation having the first wavelength comprises directing radiation at a subcutaneous target in the patient.

39. The method of claim 32 wherein:
irradiating the target with radiation having the first wavelength comprises activating an imaging agent in the patient so that at least a portion of the imaging agent at the target fluoresces; and
generating the digital three-dimensional image comprises capturing an image of the imaging agent fluorescing.

40. The method of claim 32 wherein irradiating the target in the patient comprises automatically scanning a laser beam across the target.

41. A system for delivering radiation therapy to treat a disorder in a patient, the system comprising:
an imaging unit for capturing an image of a target in the patient;
a laser for irradiating the target and activating a photodynamic therapy agent in the patient; and
a controller operably coupled to the imaging unit and the laser for operating the laser to irradiate the target based on the captured image of the target.

42. The system of claim 41 wherein:
the laser is configured to generate a laser beam along a beam path;
the system further comprises an optical element assembly aligned with the beam path for redirecting and/or conditioning the laser beam; and
the controller is operably coupled to the optical element assembly and configured to scan the laser beam across the target based on the captured image of the target.

43. The system of claim 41 wherein the imaging unit is configured to capture a three-dimensional image of the target.

44. The system of claim 41 wherein:
the laser comprises a first laser; and
the system further comprises a second laser for irradiating the target to activate an imaging agent at the target.

45. The system of claim 41 wherein:
the laser comprises a first laser for irradiating the target with radiation having a first wavelength; and
the system further comprises a second laser for irradiating the target with radiation having a second wavelength different than the first wavelength.

46. The system of claim 41 wherein:
the laser comprises a first laser for irradiating the target and activating the photodynamic therapy agent; and
the system further comprises a second laser for irradiating the target and activating an imaging agent.

47. The system of claim 41 wherein the imaging unit comprises a camera.

48. The system of claim 41 wherein the laser comprises a pulsed laser capable of initiating efficient two-photon absorption.

49. The system of claim 41 wherein the controller comprises a computer-readable medium containing instructions to perform a method comprising:
capturing the image of the target in the patient; and
directing the laser beam at the target based on the captured image.

50. The system of claim 41 wherein the controller comprises a computer-readable medium containing instructions to perform a method comprising scanning the laser beam across the target based on the captured image of the target.

51. A system for delivering radiation therapy to treat a disorder in a patient, the system comprising:
a first radiation delivery device for irradiating a target in the patient with radiation having a first wavelength;
an imaging unit for capturing a digital image of the target;
a second radiation delivery device for irradiating the target with radiation having a second wavelength different than the first wavelength; and
a controller operably coupled to the second radiation delivery device and the imaging unit for irradiating the target with radiation having the second wavelength based on the captured digital image.

52. The system of claim 51 wherein:
the first radiation delivery device comprises a first laser; and
the second radiation delivery device comprises a second laser.

53. The system of claim 51 wherein:
the second radiation delivery device comprises a laser configured to generate a laser beam along a beam path;
the system further comprises an optical element assembly aligned with the beam path for redirecting and/or conditioning the laser beam; and
the controller is operably coupled to the optical element assembly and configured to scan the laser beam across the target based on the captured image of the target.

54. The system of claim 51 wherein the second radiation delivery device comprises a laser configured to generate a laser beam along a beam path, and wherein the controller comprises a computer-readable medium containing instructions to perform a method comprising:
capturing the image of the target in the patient; and
irradiating the target with the laser beam directed at the target based on the captured image.

55. The system of claim 51 wherein:
the second radiation delivery device comprises a laser configured to generate a laser beam along a beam path; and
the controller comprises a computer-readable medium containing instructions to perform a method comprising scanning the laser beam across the target based on the captured image of the target.

56. A system for delivering radiation therapy to treat a disorder in a patient, the system comprising:
means for capturing an image of a target in the patient;
means for irradiating the target with a radiation beam; and
means for controlling the irradiation of the target based on the captured image of the target.

57. The system of claim 56 wherein the means for irradiating the target comprise a pulsed laser capable of initiating efficient two-photon absorption.

58. The system of claim 56 wherein the means for capturing the image comprise an imaging unit configured to capture a digital three-dimensional image.

59. The system of claim 56 wherein:
the means for irradiating the target comprise a laser configured to generate a laser beam along a beam path;
the system further comprises means for redirecting and/or conditioning the laser beam;
the means for controlling the irradiation comprise a controller operably coupled to the means for redirecting and/or conditioning the laser beam; and
the controller is configured to scan the laser beam across the target based on the captured image of the target.

60. The system of claim 56 wherein:
the means for irradiating the target comprise a laser configured to generate a laser beam along a beam path; and
the controller comprises a computer-readable medium containing instructions to perform a method comprising scanning the laser beam across the target based on the captured image of the target.

61. The system of claim 56 wherein:
the means for irradiating the target comprise means for irradiating the target with radiation at a first wavelength; and
the system further comprises means for irradiating the target with radiation at a second wavelength different than the first wavelength.
